(19) United States
(12) Patent Application Publication
(10) Pub. No.: US 2026/0357238 A1
(43) Pub. Date: Nov. 12, 2026

(54) METHOD FOR PREPARING HIGH-ACTIVITY NATTOKINASE POWDER

(71) Applicant: CONTEK LIFE SCIENCE CO., LTD., Taipei City (TW)

(72) Inventors: Dai-Chi WU, Taipei City (TW); James YU, Taipei City (TW); Yueh-Chi HSU, Taipei City (TW); Hsin-Te CHEN, Taipei City (TW)

(73) Assignee: CONTEK LIFE SCIENCE CO., LTD., Taipei City (TW)

(21) Appl. No.: 19/203,781

(22) Filed: May 9, 2025

(51) Int. Cl. C12N 9/64; C12P 21/02; C12R 1/125

(52) U.S. Cl. CPC C12N 9/6424; C12P 21/02; C12R 2001/125; C12Y 304/21062

(57) ABSTRACT

A method for preparing a high-activity nattokinase powder of the present invention uses Bacillus subtilis for culture and ferments to obtain a nattokinase. The method includes an activation step, a first-stage fermentation step, a second-stage fermentation step, a centrifugation step, a filtration step, and a lyophilization step. In the first-stage fermentation step and the second-stage fermentation step, a new fed-batch feeding formula is used for providing for fermentation with Bacillus subtilis, where the feed formula includes a plurality of carbon sources, a plurality of nitrogen sources, a plurality of inorganic salts and a plurality of supplementary nutritional sources, where the carbon source includes maltodextrin, the supplementary nutritional source includes a rice bran extract, the nattokinase and manganese yeast, making the final nattokinase powder has an activity value greater than 80,000 FU/g.

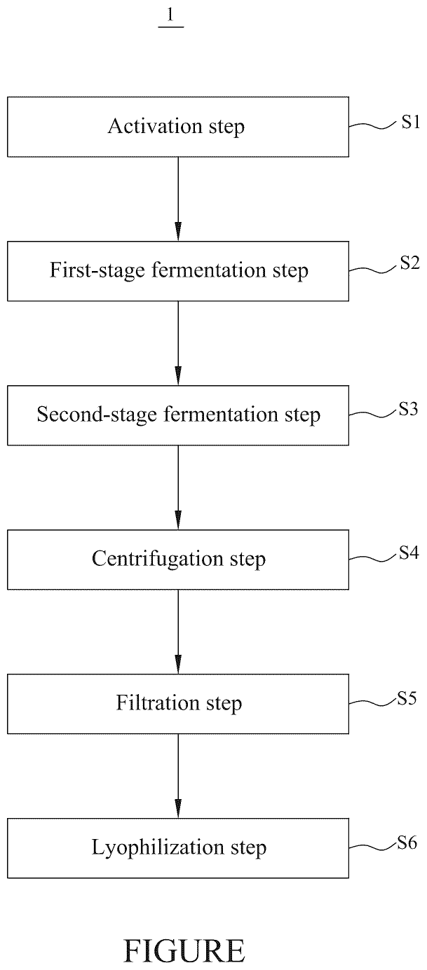

### FIELD OF THE INVENTION

The present invention relates to a method for preparing high-activity nattokinase powder, and in particular to a preparation method in which the activity of the finally produced nattokinase powder is improved by changing a formula ratio.

### BACKGROUND OF THE INVENTION

A nattokinase is a serine protease produced by Bacillus subtilis in a process of soybean fermentation. Since the nattokinase has the effects of promoting thrombolysis, improving blood circulation and lowering blood pressure, it is widely used in the fields of health-care food and medicine. In the prior art, the preparation of the nattokinase mainly relies on liquid fermentation technology. However, due to the influence of factors such as fermentation conditions, culture medium formulas, enzyme purification methods, etc., current technology still has many challenges in improving the activity value (FU/g, Fibrinolytic Units per gram) of the nattokinase.

The activity value is an important indicator to measure the potency of the nattokinase, usually expressed in FU/g. The thrombolytic ability is stronger at the same dose when the activity value is higher. Therefore, improving the activity value of the nattokinase is very important for improving the effect and market competitiveness of the product. However, the prior art has many limitations in terms of improving the activity value. For example, the selected strains have different requirements for the fermentation environments, which leads to differences in the activity values of the products. Moreover, the selection of culture conditions affects the synthesis and secretion of enzymes, where the types and proportions of carbon sources, nitrogen sources and inorganic salts will also affect the synthesis and secretion of the nattokinase.

The carbon source is a main energy source for microbial growth and metabolism. Different types of carbon sources (e.g. dextrose, sucrose or maltose) will affect the growth rate of bacteria and enzyme production. The nitrogen source provides the amino acids and proteins required for biosynthesis. The nitrogen source can be divided into organic nitrogen (e.g. yeast powder and peptone) and inorganic nitrogen (e.g. ammonium nitrate and ammonium sulfate). Choosing a suitable nitrogen source can promote the production of the nattokinase. The inorganic salt affects the metabolic balance of cells and the stability of enzyme activity. For example, calcium ions (Ca²⁺) and magnesium ions (Mg²⁺) facilitate maintaining the activity of proteases, while excessive metal ions may cause an inhibition effect on enzymes. Therefore, it is a problem that the prior art needs to solve how to accurately regulate the types and concentrations of carbon sources, nitrogen sources and inorganic salts to improve the activity value of the nattokinase.

Moreover, in addition to the fermentation conditions, dissolved oxygen and a culture pH value will also affect the activity of the nattokinase. Proper oxygen supply and pH value regulation can improve the stability of the enzyme and avoid a decrease in the activity due to protein degradation.

Furthermore, purification and drying will also affect the activity value of the final product. Traditional purification and drying techniques, e.g. high-temperature drying or spray drying, may cause structural denaturation of the nattokinase and thus reduce the activity of the nattokinase. As a result, the activity values of most nattokinase products on the market are only between 10,000-40,000 FU/g. It has become a problem that needs to be overcome in the prior art how to improve the activity of the nattokinase through appropriate formula ratios, culture conditions, centrifugation conditions, filtration conditions and drying technology.

### SUMMARY OF THE INVENTION

A main objective of the present invention is to make the activity value of finally produced nattokinase powder be greater than 80,000 FU/g by changing the formula ratio of a fed-batch feeding culture solution.

A secondary objective of the present invention is to make the activity value of the finally produced nattokinase powder be greater than 80,000 FU/g by changing the formula ratio of an excipient during lyophilization.

To achieve the aforementioned objectives, a method for preparing high-activity nattokinase powder of the present invention includes:
(a) an activation step: inoculating a Bacillus subtilis into a TSB medium, waiting for the Bacillus subtilis to be activated to obtain a first strain culture solution, and measuring an OD600 value of the first strain culture solution with a spectrophotometer so that the value is between 0.8 to 1.5;
(b) a first-stage fermentation step: adding the first strain culture solution into a fed-batch feeding culture solution, and then placing into a fermentation tank and culturing for 36-48 hours to obtain a second strain culture solution;
(c) a second-stage fermentation step: adding the second strain culture solution into the fed-batch feeding culture solution, and then placing into the fermentation tank and culturing for 12-48 hours to obtain a third strain culture solution;
(d) a centrifugation step: subjecting the third strain culture solution to centrifugal treatment to obtain a fermentation solution after separation;
(e) a filtering step: subjecting the fermentation solution to filtering treatment to obtain a filtrate after separation, where a protein concentration of the filtrate is 1.33%-2.67% (w/v); and
(f) a lyophilization step: mixing the filtrate with an excipient and lyophilizing for 40-65 hours to obtain nattokinase powder, where the nattokinase powder has a moisture content <5%, a water activity value <1A_{w}, and an activity value greater than 80,000 FU/g.

The fed-batch feeding culture solution includes a plurality of carbon sources, a plurality of nitrogen sources, a plurality of inorganic salts and a plurality of supplementary nutritional sources. The plurality of carbon sources include maltodextrin, and the plurality of supplementary nutritional sources include a rice bran extract, nattokinase and a manganese yeast.

The plurality of carbon sources include 0.1-10 parts of maltodextrin, 0.5-10 parts of sucrose, and 0.01-5 parts of dextrose. The plurality of nitrogen sources include 0.5-10 parts of soy powder and 0.1-5 parts of a yeast extract. The plurality of inorganic salts include 0.005-3 parts of disodium hydrogen phosphate (Na₂HPO₄), 0.005-2 parts of dipotassium hydrogen phosphate (K₂HPO₄), 0.005-2 parts of magnesium sulfate (MgSO₄), 0.01-1 parts of potassium dihydrogen phosphate (KH₂PO₄) and 0.001-1 parts of calcium chloride (CaCl₂)). The plurality of supplementary nutritional sources include 0.01-5 parts of the rice bran extract, 0.005-2 parts of the nattokinase, 0.001-1 parts of the manganese yeast and 0.001-1 parts of the magnesium yeast.

The plurality of carbon sources include 1.5-4.5 parts of maltodextrin, 2-7 parts of sucrose, and 0.2-2 parts of dextrose. The plurality of nitrogen sources include 2-7 parts of soy powder and 0.5-2.5 parts of a yeast extract. The plurality of inorganic salts include 0.05-1 parts of disodium hydrogen phosphate (Na₂HPO₄), 0.05-1 parts of dipotassium hydrogen phosphate (K₂HPO₄), 0.05-1 parts of magnesium sulfate (MgSO₄), 0.05-0.4 parts of potassium dihydrogen phosphate (KH₂PO₄) and 0.01-0.15 parts of calcium chloride (CaCl₂)). The plurality of supplementary nutritional sources include 0.2-1.5 parts of the rice bran extract, 0.05-1 parts of the nattokinase, 0.01-0.1 parts of the manganese yeast and 0.01-0.1 parts of the magnesium yeast.

The fed-batch feeding culture solution further includes 0.01-1 parts of a soy defoamer.

Moreover, in the activation step, while waiting for the Bacillus subtilis to be activated, the first container is shaken at 150-250 rpm for 3-10 hours.

Additionally, in the activation step, while waiting for the Bacillus subtilis to be activated, samples are taken at 3, 5, and 7 hours after the activation to measure OD600 values. If the value is less than 0.05, 0.01%-0.05% of the first volume of the Bacillus subtilis strain solution is additionally added into the first container.

Furthermore, the first container has a first volume, the second container has a second volume, and the third container has a third volume, where the second volume is 20-500 times larger than the first volume, and the third volume is 10-1,000 times larger than the second volume. In the first-stage fermentation step, the first strain culture solution and the fed-batch feeding culture solution together account for 30%-80% of the second volume; and in the second-stage fermentation step, the second strain culture solution and the fed-batch feeding culture solution together account for 30%-70% of the third volume.

Moreover, the culturing temperatures of the activation step, the first-stage fermentation step and the second-stage fermentation step are 35-40 °C.

Furthermore, during the culture process of the first-stage fermentation step and the second-stage fermentation step, the pH value is maintained at 6.5 by using sodium hydroxide and hydrochloric acid, and the dissolved oxygen (DO) is maintained at 5%-50%.

In this embodiment, in the culture process of the first-stage fermentation step, after 5-10 hours, the fed-batch feeding culture solution is continuously added into the second container using a peristaltic pump for a total fed-batch feeding time of 3-18 hours; and in the culture process of the second-stage fermentation step, after 3-12 hours, the fed-batch feeding culture solution is continuously added into the third container using the peristaltic pump for a total fed-batch feeding time of 3-18 hours.

In the centrifugation step, the centrifugation conditions are centrifuging at 12,000 rpm and 3-18 °C for 15-60 minutes before taking out.

Moreover, the excipient includes 8-75 parts of maltodextrin or resistant dextrin mixed with 30-70 parts of trehalose, 15-40 parts of mannitol, 5-15 parts of sorbitol, 5-15 parts of sucrose, 5-15 parts of glycine, 1-10 parts of lactose and 1-10 parts of dextran.

The lyophilization step includes a freezing treatment stage, a primary drying stage and a secondary drying stage sequentially. The freezing treatment stage is freezing at a temperature of −40 °C-0 °C for 2-12 hours. The primary drying stage is slowly raising the temperature from 0 °C to 30 °C for a total time of 10-25 hours after the vacuum pressure is less than 100 mTorr. The secondary drying stage is drying at a temperature of 30 °C-45 °C for 8-15 hours.

The present invention is characterized in that the fed-batch feeding culture solution includes the plurality of carbon sources, the plurality of nitrogen sources, the plurality of inorganic salts and the plurality of supplementary nutritional sources, where the carbon source includes the maltodextrin, and the supplementary nutritional source include the rice bran extract, the nattokinase and the manganese yeast, so that the activity value of the final nattokinase powder is greater than 80,000 FU/g.

Moreover, the excipient includes 30-70 parts of the trehalose, 15-40 parts of the mannitol, 5-15 parts of the sorbitol, 5-15 parts of the sucrose, 5-15 parts of the glycine, 1-10 parts of the lactose and 1-10 parts of the dextran, so that the activity value of the final nattokinase powder is greater than 80,000 FU/g.

### BRIEF DESCRIPTION OF THE DRAWINGS

The FIGURE is a schematic flow diagram of a method for preparing high-activity nattokinase powder of the present invention.

### DETAILED DESCRIPTION OF THE PREFERRED EMBODIMENTS

In order to have further clear and detailed knowledge and understanding of the construction, use and features of the invention, preferred embodiments are given and described in detail with reference to the drawings as follows:

Unless otherwise specified, the term “nattokinase” as used herein refers to a serine protease produced by Bacillus subtilis, which has fibrinolytic activity, with the activity unit being expressed in FU/g.

Unless otherwise specified, the term “activity value (FU/g)” as used herein refers to the fibrinolytic activity of the nattokinase per gram of a product. The potency of the enzyme is stronger when the value is higher. In the present invention, “high activity” refers to an activity value greater than 80,000 FU/g.

Unless otherwise specified, the term “carbon source” as used herein refers to a component in a culture medium that provides energy for metabolism, including but not limited to sucrose and dextrose, the appropriate ratio of which can affect the fermentation efficiency and product activity.

Unless otherwise specified, the term “nitrogen source” as used herein refers to a nutrient required for cell protein synthesis, including soy powder and a yeast extract. The combination of different nitrogen sources can affect the production and activity of the nattokinase.

Unless otherwise specified, the term “inorganic salts” as used herein refers to inorganic salts that provide trace elements to promote cell growth during the fermentation process, including disodium hydrogen phosphate, dipotassium hydrogen phosphate, magnesium sulfate, potassium dihydrogen phosphate and calcium chloride.

Unless otherwise specified, the term “lyophilization” as used herein refers to a drying method that powders the nattokinase by freezing and removing water under reduced pressure, which can protect the activity of the enzyme and improve its stability.

Unless otherwise specified, the term “excipient” as used herein refers to auxiliary substances added to protect the activity of the nattokinase and improve the properties of the powder, including but not limited to maltodextrin, resistant dextrin, trehalose, mannitol, etc.

Unless otherwise specified, the term “activation” as used herein refers to resuscitating a strain from a deposition state (e.g. frozen, lyophilized or stored in glycerol) and culturing it to an appropriate growth state to improve cell activity and fermentation efficiency, which are usually accomplished through liquid or solid culture.

Unless otherwise specified, the term “fermentation” as used herein refers to a process in which microorganisms such as Bacillus subtilis grow and produce the nattokinase under specific conditions.

Unless otherwise specified, the term “OD600 (Optical Density at 600 nm)” as used herein refers to the optical density of a culture solution at a wavelength of 600 nm determined with a spectrophotometer to estimate the concentration of microbial cells. Generally, a higher value indicates a higher concentration of cells in the culture solution.

Unless otherwise specified, the term “fed-batch feeding” as used herein refers to the continuous or intermittent addition of carbon sources, nitrogen sources or trace elements in a fermentation process to maintain microbial survival and improve microbial growth efficiency and enzyme production, and to avoid substrate inhibition or nutrient depletion.

Unless otherwise specified, the term “dissolved oxygen (DO)” as used herein refers to the concentration of dissolved oxygen in the fermentation solution, which affects the growth and metabolism of microorganisms.

Unless otherwise specified, the term “agitation gradient” as used herein refers to the way the rotation speed of a stirring blade in a fermentation tank changes, which can affect the oxygen and nutrient distribution of the culture medium. Usually, the strain growth and enzyme secretion are controlled by gradually increasing or decreasing the rotation speed.

Unless otherwise specified, the term “centrifugation” as used herein refers to the use of high-speed rotation to generate a centrifugal force to separate the strains, cell debris and soluble proteins in the fermentation solution, ensuring the efficiency of subsequent purification steps.

Unless otherwise specified, the term “filtration” as used herein refers to the use of membrane filtration technology (e.g. microfiltration or ultrafiltration) to separate the nattokinase from impurities, so as to avoid protein denaturation and improve purity.

Unless otherwise specified, the term “water activity (A_{w})” as used herein refers to the free water content inside a product related to the potential risk of microbial growth.

Unless otherwise specified, the term “moisture content” as used herein refers to the weight percentage (%) of moisture in the product, which affects the flowability and storage stability of the enzyme powder.

Unless otherwise specified, the term “refractive index” as used herein refers to the degree to which light is refracted in a sample, reflecting the concentration and composition of a solution.

Unless otherwise specified, the term “supernatant” as used herein refers to a clear liquid formed above a solid pellet after centrifugation treatment, which may contain soluble proteins, metabolites, enzymes (e.g. the nattokinase), culture medium residues and other water-soluble components.

Unless otherwise specified, the term “microfiltration (MF)” as used herein refers to the technology of filtering using a filter membrane with a pore size in a range of about 0.1-10 microns (μm), which can mainly remove suspended particles, strain cells and other large-sized impurities in a liquid without affecting the penetration of soluble proteins or small molecular metabolites.

Unless otherwise specified, the term “ultrafiltration (UF)” as used herein refers to the technology of selective filtration using a filter membrane with a pore size in a range of about 1-100 nanometers (nm). Compared with microfiltration, the ultrafiltration can filter proteins, enzymes (e.g. the nattokinase) and polymer compounds with larger molecular weights, while allowing small-molecule substances (e.g. inorganic salts and small-molecular metabolites) to pass through the filter membrane.

Unless otherwise specified, the term “magnesium yeast” as used herein refers to the addition of magnesium ion sources (e.g., inorganic magnesium salts such as magnesium oxide and magnesium sulfate) in the process of yeast culture, which are absorbed and biotransformed by a yeast to form organic magnesium-containing yeast cell substances. The magnesium yeast can provide bioavailable magnesium elements, which serve as metal cofactors required for enzyme reactions in the fermentation process, thereby promoting the yield and activity performances of target enzymes (e.g., the nattokinase).

Unless otherwise specified, the term “manganese yeast” as used herein refers to organic manganese-containing yeast cell substances produced by adding a manganese ion source (e.g., manganese sulfate) in the yeast culture process, which is absorbed, metabolized and transformed by the yeast. The organic manganese contained in manganese yeast can serve as a coenzyme component of certain metal enzymes, enhance the antioxidant capacity and metabolic efficiency of cells, and thus improve the fermentation performance and quality stability of the target product.

Referring to the FIGURE, in a first preferred example, a method 1 for preparing high-activity nattokinase powder of the present invention included an activation step S1, a first-stage fermentation step S2, a second-stage fermentation step S3, a centrifugation step S4, a filtration step S5 and a lyophilization step S6, where:
(a) the activation step S1: a liquid TSB medium was added into a first container having a first volume, and a Bacillus subtilis was inoculated into the TSB medium, it was waited for the Bacillus subtilis to be activated to obtain a first strain culture solution, and an OD600 value of the first strain culture solution was measured by using a spectrophotometer, so that the value was between 0.8 to 1.5;
(b) the first-stage fermentation step S2: the first strain culture solution was added into a second container having a second volume, added with a fed-batch feeding culture solution, and then placed into a fermentation tank and cultured for 36-48 hours to obtain a second strain culture solution;
(c) the second-stage fermentation step S3: the second strain culture solution was added into a third container having a third volume, added with a fed-batch feeding culture solution, and then placed into a fermentation tank and cultured for 12-48 hours to obtain a third strain culture solution;
(d) the centrifugation step S4: the third strain culture solution was subjected to centrifugal treatment to obtain a fermentation solution after separation;
(e) the filtration step S5: the fermentation solution was filtered to obtain a filtrate after separation, where the filtrate had a refractive index of 1.339-1.357 at 4-28 °C and a protein concentration of 1.33%-2.67% (w/v); and
(f) the lyophilization step: the filtrate was mixed with an excipient and lyophilized for 40-65 hours to obtain nattokinase powder, where the nattokinase powder had a moisture content <5%, a water activity value <1A_{w}, and an activity value greater than 80,000 FU/g.

The fed-batch feeding culture solution included a plurality of carbon sources, a plurality of nitrogen sources, a plurality of inorganic salts and a plurality of supplementary nutritional sources. The carbon source included 0.1-10 parts of maltodextrin, and the supplementary nutritional source included 0.01-5 parts of a rice bran extract, 0.005-2 parts of a nattokinase and 0.001-1 parts of a manganese yeast.

In the activation step S1, firstly the liquid TSB medium was added into the first container, and then the Bacillus subtilis was inoculated into the TSB medium; and then, the first container was placed into an incubator shaker and shaken at 150-250 rpm for 3-10 hours, and the culture temperature was maintained at 35-°C, waiting for the activation of the Bacillus subtilis.

The TSB medium was a common trypticase soy medium on the market, and its formula mainly included: tryptone, soy peptone, sodium chloride (NaCl), dextrose and dipotassium hydrogen phosphate (K₂HPO₄).

Furthermore, in the process of Bacillus subtilis activation, a portion of the first strain culture solution was taken respectively at 3, 5, and 7 hours after activation, and the OD600 value was measured using the spectrophotometer to confirm whether the strain concentration reached a target value of 0.8-1.5. If the value was less than 0.05, then 0.01%-0.05% of the first volume of the Bacillus subtilis culture solution was additionally added into the first container until the value reached the target value between 0.8-1.5.

In the first-stage fermentation step S2, firstly the second container was prepared with the second volume of the second container being 20-500 times larger than the first volume of the first container; then, the first strain culture solution after the activation step S1 was added into the second container, and added with the fed-batch feeding culture solution for mixing with the first strain culture solution, so that the first strain culture solution and the fed-batch feeding culture solution together accounted for 30%-80% of the second volume; and finally, the second container was placed into the fermentation tank and cultured for 36-48 hours to obtain the second strain culture solution.

During the culture process, the culture temperature was maintained between 35-40 °C, and the pH value was maintained at 6.5 using sodium hydroxide and hydrochloric acid. Finally, the rotation speeds of the fermentation tank at different times were controlled according to Table 1 below, and oxygen was continuously supplied to maintain the dissolved oxygen (DO) at 5%-50%.

TABLE 1

| Time | Rotation speed (rpm) | |
|---|---|---|
| 0-10th hour | 80-300 | rpm |
| 6-30th hour | 100-800 | rpm |
| 24-48th hour | 150-1,200 | rpm |

Also, in the culture process, after 5-10 hours, the fed-batch feeding culture solution was continuously added into the second container using the peristaltic pump for a total fed-batch feeding time of 3-18 hours, so that the Bacillus subtilis had sufficient nutrients to ferment.

In the second-stage fermentation step S3, the third container was first prepared with the third volume of the third container being 10-1,000 times larger than the second volume of the second container; then, the second strain culture solution after the first-stage fermentation step S2 was added into the third container, and added with the fed-batch feeding culture solution for mixing with the second strain culture solution, so that the second strain culture solution and the fed-batch feeding culture solution together accounted for 30%-70% of the third volume; and finally, the third container was placed into the fermentation tank and cultured for 12-48 hours to obtain the third strain culture solution.

During the culture process, the culture temperature was maintained between 35-40 °C, and the pH value was maintained at 6.5 using sodium hydroxide and hydrochloric acid. Finally, the rotation speeds of the fermentation tank at different times were controlled according to Table 1 above, and oxygen was continuously supplied to maintain the dissolved oxygen (DO) at 5%-50%.

Also, in the culture process, after 3-12 hours, the fed-batch feeding culture solution was continuously added into the third container using the peristaltic pump for a total fed-batch feeding time of 3-18 hours, so that the Bacillus subtilis had sufficient nutrients to ferment.

In this example, the composition formulas and proportions of the fed-batch feeding culture solution were shown in Table 2 below.

TABLE 2

| | Weight percentage concentration |
|---|---|
| Components | (% w/v) |
| Sucrose | 0.5-10% |
| Soy powder | 0.5-10% |
| Maltodextrin | 0.1-10% |
| Yeast extract | 0.1-5% |
| Dextrose | 0.01-5% |
| Rice bran extract | 0.01-5% |
| Disodium hydrogen phosphate | 0.005-3% |
| (Na₂HPO₄) | |
| Dipotassium hydrogen phosphate | 0.005-2% |
| (K₂HPO₄) | |
| Magnesium sulfate (MgSO₄) | 0.005-2% |
| Nattokinase | 0.005-2% |
| Potassium dihydrogen phosphate | 0.01-1% |
| (KH₂PO₄) | |
| Soy defoamer | 0.01-1% |
| Calcium chloride (CaCl₂) | 0.001-1% |
| Manganese yeast | 0.001-1% |
| Magnesium yeast | 0.001-1% |

The balance of deionized water

Among them, sucrose, maltodextrin and dextrose were carbon sources of the fed-batch feeding culture solution, which could provide the energy required for the growth and reproduction of the Bacillus subtilis and the carbon components necessary for the synthesis of strains.

The soy powder and the yeast extract were nitrogen sources of the fed-batch feeding culture solution, which could provide the nutrients required for the Bacillus subtilis to synthesize the nattokinase.

Among them, disodium hydrogen phosphate, dipotassium hydrogen phosphate, magnesium sulfate, potassium dihydrogen phosphate and calcium chloride were inorganic salts of the fed-batch feeding culture solution, which could provide trace elements to the Bacillus subtilis to promote the growth of the Bacillus subtilis and synthesis of the nattokinase.

The rice bran extract, the nattokinase, the manganese yeast and the magnesium yeast were supplementary nutritional sources of the fed-batch feeding culture solution, which could promote the Bacillus subtilis to synthesize the nattokinase.

In this example, the soy defoamer as used was Kureton Power produced by Kao Corporation, Japan, the main components of which included fatty acid glycerides, lecithin, magnesium carbonate (MgCO₃) and silicone resin, and it could inhibit the foaming of the first strain culture solution, the second strain culture solution and the third strain culture solution in the culture process, thereby causing the experimental environment to be dirty and disorderly.

However, the soy defoamer could also be other types of defoamers as long as they could achieve the purpose of eliminating foam.

In the centrifugation step S4, the third strain culture solution that had subjected to the second-stage fermentation step S3 was subjected to centrifugal treatment using a centrifuge with the centrifugation conditions being set to centrifuging at 12,000 rpm and 3-18 °C for 15-60 minutes, and then taking out, and the supernatant was taken out to obtain a fermentation solution. After the centrifugation step S4, most of the strains and the metabolic residues and impurities in the third strain culture solution could be removed.

In the filtration step S5, the fermentation solution after the centrifugation step S4 was sequentially subjected to microfiltration treatment and ultrafiltration treatment. In the microfiltration treatment, a first thin membrane having a first pore size was used for microfiltration, and in the ultrafiltration treatment, a second thin membrane having a second pore size was used for ultrafiltration. Finally, the filtrate was obtained, where the second pore size was smaller than the first pore size, so that the strains and macromolecular impurities that were not removed in the centrifugation step S4 could be removed from the fermentation solution after the microfiltration treatment, and the remaining small molecular impurities could be removed from the fermentation solution after the ultrafiltration treatment, thereby improving the purity of the product.

The filtrate after the filtration step S5 had a refractive index of 1.339-1.357 at 4-28 °C, and a protein concentration of 1.33%-2.67% (w/v).

In the lyophilization step S6, the filtrate after the filtering step S5 was mixed with the excipient and placed into a lyophilizer for lyophilization for 40-65 hours to obtain the nattokinase powder. The lyophilization step included a freezing treatment stage, a primary drying stage and a secondary drying stage sequentially. The freezing treatment stage was freezing at a temperature of −40 °C-0 °C for 2-12 hours. The primary drying stage was slowly raising the temperature from 0 °C to °C for a total time of 10-25 hours after the vacuum pressure was less than 100 mTorr. The secondary drying stage was drying at a temperature of 30 °C-45 °C for 8-15 hours. The excipient included 8-75 parts of maltodextrin or resistant dextrin mixed with 30-70 parts of trehalose, 15-40 parts of mannitol, 5-15 parts of sorbitol, 5-15 parts of sucrose, 5-15 parts of glycine, 1-10 parts of lactose and 1-10 parts of dextran.

Also, the nattokinase powder after the lyophilization step S6 had the moisture value of <5%, and the water activity value of <1A_{w}, and the nattokinase powder was also sequentially subjected to grinding, screening, and magnet selection to further improve the quality, solubility and safety of the final product.

In a first preferred comparative example, the only difference from the first preferred example was the different formula of the fed-batch feeding culture solution, and the rest of the methods and experimental conditions were the same as those of the first preferred example, so they were not described in detail any more.

The composition formulas and proportions of the fed-batch feeding culture solution were shown in Table 3 below.

TABLE 3

| | Weight percentage concentration |
|---|---|
| Components | (% w/v) |
| Sucrose | 0.5-10% |
| Soy powder | 0.5-10% |
| Yeast extract | 0.1-5% |
| Dextrose | 0.01-5% |
| Disodium hydrogen phosphate | 0.005-3% |
| (Na₂HPO₄) | |
| Dipotassium hydrogen phosphate | 0.005-2% |
| (K₂HPO₄) | |
| Magnesium sulfate (MgSO₄) | 0.005-2% |
| Nattokinase | 0.005-2% |
| Potassium dihydrogen phosphate | 0.01-1% |
| (KH₂PO₄) | |
| Soy defoamer | 0.01-1% |
| Calcium chloride (CaCl₂) | 0.001-1% |
| Magnesium yeast | 0.001-1% |

The balance of deionized water

In a second preferred comparative example, the only difference from the first preferred example was the different formula of the fed-batch feeding culture solution, and the rest of the methods and experimental conditions were the same as those of the first preferred example, so they were not described in detail any more.

The composition formulas and proportions of the fed-batch feeding culture solution were shown in Table 4 below.

TABLE 4

| | Weight percentage |
|---|---|
| Components | concentration (% w/v) |
| Sucrose | 0.5-10% |
| Soy powder | 0.5-10% |
| Yeast extract | 0.1-5% |
| Dextrose | 0.01-5% |
| Rice bran extract | 0.01-5% |
| Disodium hydrogen phosphate (Na₂HPO₄) | 0.005-3% |
| Dipotassium hydrogen phosphate | 0.005-2% |
| (K₂HPO₄) | |
| Magnesium sulfate (MgSO₄) | 0.005-2% |
| Potassium dihydrogen phosphate | 0.01-1% |
| (KH₂PO₄) | |
| Soy defoamer | 0.01-1% |
| Calcium chloride (CaCl₂) | 0.001-1% |
| Manganese yeast | 0.001-1% |
| Magnesium yeast | 0.001-1% |

The balance of deionized water

In a third preferred comparative example, the only difference from the first preferred example was the different formula of the fed-batch feeding culture solution, and the rest of the methods and experimental conditions were the same as those of the first preferred example, so they were not described in detail any more.

The composition formulas and proportions of the fed-batch feeding culture solution were shown in Table 5 below.

TABLE 5

| | Weight percentage |
|---|---|
| Components | concentration (% w/v) |
| Sucrose | 0.5-10% |
| Soy powder | 0.5-10% |
| Maltodextrin | 0.1-10% |
| Yeast extract | 0.1-5% |
| Dextrose | 0.01-5% |
| Disodium hydrogen phosphate (Na₂HPO₄) | 0.005-3% |
| Dipotassium hydrogen phosphate | 0.005-2% |
| (K₂HPO₄) | |
| Magnesium sulfate (MgSO₄) | 0.005-2% |
| Potassium dihydrogen phosphate | 0.01-1% |
| (KH₂PO₄) | |
| Soy defoamer | 0.01-1% |
| Calcium chloride (CaCl₂) | 0.001-1% |
| Magnesium yeast | 0.001-1% |

The balance of deionized water

In a fourth preferred comparative example, the only difference from the first preferred example was the different formula of the excipient, and the rest of the methods and experimental conditions were the same as those of the first preferred example, so they were not described in detail any more.

Only trehalose was used as the excipient.

In a fifth preferred comparative example, the only difference from the first preferred example was the different formula of the excipient, and the rest of the methods and experimental conditions were the same as those of the first preferred example, so they were not described in detail any more.

The excipient included 8-75 parts of maltodextrin or 8-75 parts of resistant dextrin mixed with 30-70 parts of trehalose and 5-15 parts of glycine.

In a sixth preferred comparative example, the only difference from the first preferred example was the different formulas of the fed-batch feeding culture solution and the excipient, and the rest of the methods and experimental conditions were the same as those of the first preferred example, so they were not described in detail any more. The excipient included 8-75 parts of maltodextrin or 8-75 parts of resistant dextrin mixed with 30-70 parts of trehalose, 15-40 parts of mannitol, and 1-10 parts of dextran.

Finally, the activity value of the nattokinase powder of the first preferred example was compared with those of the nattokinase powder of the first to sixth preferred comparative examples, and the activity values were detected according to the method in U.S. Pat. No. 7,435,565 B2. The activity value results were shown in Table 6 below.

TABLE 6

| | | Comparative | Percentage of |
|---|---|---|---|
| | Examples | Examples | improvement (%) |
| The first | 91821 | 71035 | 29% |
| preferred | | | |
| comparative | | | |
| example | | | |
| The second | 95241 | 83055 | 15% |
| preferred | | | |
| comparative | | | |
| example | | | |
| The third | 76629 | 66823 | 15% |
| preferred | | | |
| comparative | | | |
| example | | | |
| The fourth | 89210 | 73025 | 22% |
| preferred | | | |
| comparative | | | |
| example | | | |
| The fifth | 89508 | 80085 | 12% |
| preferred | | | |
| comparative | | | |
| example | | | |
| The sixth | 96022 | 85913 | 12% |
| preferred | | | |
| comparative | | | |
| example | | | |

(Unit: FU/g-powder)

The method for detecting the activity value in U.S. Pat. No. 7,435,565 B2 was to allow the nattokinase to act on a fibrous protein and measure the activity of the nattokinase by the increase of acid-soluble low-molecular-weight degradation products produced by the decomposition of the fibrous protein. This increase was determined by measuring the absorbance of UV light (275 nm), where the detection method was as follows:
(a-1) preparation of a fibrinogen aqueous solution: 72 mg of fibrinogen (Sigma Corp., fibrinogen fraction I derived from bovine blood plasma, Type IS) was dissolved in 10 ml of a 50 mM borax buffer solution (pH 8.5, 150 mM NaCl) to formulate a 0.72% (w/v) fibrinogen aqueous solution.
(a-2) Preparation of a thrombin solution: Thrombin was dissolved in a 50 mM borax buffer solution to a concentration of 1,000 U/ml. When the solution was used, it was diluted with a borax buffer solution by 50 times (i.e. 20 U/ml).
(a-3) Activity assay: 1.4 ml of the 50 mM borax buffer solution and 0.4 ml of the fibrinogen aqueous solution were placed into a test tube and heated in a water bath at 37 °C±0.0.3 °C for five minutes. Then, 0.1 ml of a thrombin solution was added and stirred. The mixture was placed into a water bath and allowed to stand for 10 minutes. Then, 0.1 ml of the sample solution was added into the mixture, stirred for 5 seconds, then placed into a water bath and allowed to stand. The mixture was stirred for 5 seconds. 20 minutes and 40 minutes. The sample solution was added. At 60 minutes after the addition of the sample solution, 2 ml of a 0.2 M trichloroacetic acid solution was added, stirred, and allowed to stand for 20 minutes. The reaction mixture was centrifuged for 5 minutes. The mixture was centrifuged at 15,000×g, and the absorbance (Ar) of the supernatant was measured at 275 nm. As a control, 1.4 ml of the 50 mM borax buffer solution and 0.4 ml of the fibrinogen aqueous solution were placed into a test tube and heated in a water bath at 37 °C.±0.0.3 °C for 5 minutes. Then, 0.1 ml of a thrombin solution was added and stirred. The mixture was placed into a water bath and allowed to stand for 10 minutes. Then 2 ml of a 0.2 M trichloroacetic acid solution was added, and stirred. Next, 0.1 ml of the sample solution was added into the mixture and stirred, and then allowed to stand for 20 minutes. The reaction mixture was centrifuged for 5 minutes. The mixture was centrifuged at 15,000×g, and the absorbance (Ac) of the supernatant was measured at 275 nm. The activity of the nattokinase was determined according to the following equation: A (FU/ml)={(Ar−Ac)/(0.01×60×0.1)}×D.

In view of the above, in the present invention the formulas of the fed-batch feeding culture solution and the excipient are changed, so that the average activity value of the nattokinase powder of the final product is greater than 80,000 FU/g, which is increased by 12%-29% compared to that of a traditional formula.

What is claimed is:

1. A method for preparing high-activity nattokinase powder, comprising:
(a) an activation step: inoculating a Bacillus subtilis into a TSB medium, waiting for the Bacillus subtilis to be activated to obtain a first strain culture solution, and measuring a value of the first strain culture solution with a spectrophotometer according to OD600; stopping activate the first strain culture solution when the value is measured between 0.8 to 1.5;
(b) a first-stage fermentation step: adding a fed-batch feeding culture solution into the first strain culture solution, and then placing into a fermentation tank and culturing for 36-48 hours to obtain a second strain culture solution;
(c) a second-stage fermentation step: adding the fed-batch feeding culture solution into the second strain culture solution, and then placing into the fermentation tank and culturing for 12-48 hours to obtain a third strain culture solution;
(d) a centrifugation step: subjecting the third strain culture solution to centrifugal treatment to obtain a fermentation solution after separation;
(e) a filtering step: subjecting the fermentation solution to filtering treatment to obtain a filtrate after separation, wherein a protein concentration of the filtrate is 1.33%-2.67% (w/v); and
(f) a lyophilization step: mixing the filtrate with an excipient and lyophilizing for 40-65 hours to obtain nattokinase powder, wherein the nattokinase powder has a moisture content <5%, a water activity value <1A_{w}, and an activity value greater than 80,000 FU/g;
wherein the fed-batch feeding culture solution comprises a plurality of carbon sources, a plurality of nitrogen sources, a plurality of inorganic salts and a plurality of supplementary nutritional sources, wherein the carbon sources comprise maltodextrin, and the supplementary nutritional sources comprise a rice bran extract, nattokinase and a manganese yeast.

2. The method for preparing high-activity nattokinase powder according to claim 1, wherein the carbon sources comprise 0.1-10 parts of maltodextrin, 0.5-10 parts of sucrose, and 0.01-5 parts of dextrose.

3. The method for preparing high-activity nattokinase powder according to claim 2, wherein the carbon sources comprise 1.5-4.5 parts of maltodextrin, 2-7 parts of sucrose, and 0.2-2 parts of dextrose.

4. The method for preparing high-activity nattokinase powder according to claim 1, wherein the nitrogen sources comprise 0.5-10 parts of soy powder and 0.1-5 parts of a yeast extract.

5. The method for preparing high-activity nattokinase powder according to claim 4, wherein the nitrogen sources comprise 2-7 parts of soy powder and 0.5-2.5 parts of a yeast extract.

6. The method for preparing high-activity nattokinase powder according to claim 1, wherein the inorganic salts comprise 0.005-3 parts of disodium hydrogen phosphate (Na₂HPO₄), 0.005-2 parts of dipotassium hydrogen phosphate (K₂HPO₄), 0.005-2 parts of magnesium sulfate (MgSO₄), 0.01-1 parts of potassium dihydrogen phosphate (KH₂PO₄) and 0.001-1 parts of calcium chloride (CaCl₂)).

7. The method for preparing high-activity nattokinase powder according to claim 6, wherein the inorganic salts comprise 0.05-1 parts of disodium hydrogen phosphate (Na₂HPO₄), 0.05-1 parts of dipotassium hydrogen phosphate (K₂HPO₄), 0.05-1 parts of magnesium sulfate (MgSO₄), 0.05-0.4 parts of potassium dihydrogen phosphate (KH₂PO₄), and 0.01-0.15 parts of calcium chloride (CaCl₂).

8. The method for preparing high-activity nattokinase powder according to claim 1, wherein the supplementary nutritional sources comprise 0.01-5 parts of a rice bran extract, 0.005-2 parts of nattokinase, 0.001-1 parts of a manganese yeast and 0.001-1 parts of a magnesium yeast.

9. The method for preparing high-activity nattokinase powder according to claim 8, wherein the supplementary nutritional sources comprise 0.2-1.5 parts of a rice bran extract, 0.05-1 parts of nattokinase, 0.01-0.1 parts of a manganese yeast, and 0.01-0.1 parts of a magnesium yeast.

10. The method for preparing high-activity nattokinase powder according to claim 1, wherein the fed-batch feeding culture solution further comprises 0.01-1 parts of a soy defoamer.

11. The method for preparing high-activity nattokinase powder according to claim 1, wherein in the activation step, while waiting for the Bacillus subtilis to be activated, the TSB medium is shaken at 150-250 rpm for 3-10 hours.

12. The method for preparing high-activity nattokinase powder according to claim 1, wherein in the activation step, while waiting for the Bacillus subtilis to be activated, samples are taken at 3, 5, and 7 hours after the activation to measure OD600 values, wherein if the value is less than 0.05, a strain solution of the Bacillus subtilis is additionally added into the TSB medium.

13. The method for preparing high-activity nattokinase powder according to claim 1, wherein in the activation step, the TSB medium and the Bacillus subtilis are activated in a first container having a first volume; in the first-stage fermentation step, the first strain culture solution and the fed-batch feeding culture solution are fermented in a second container having a second volume; and in the second-stage fermentation step, the second strain culture solution and the fed-batch feeding culture solution are fermented in a third container having a third volume, the second volume is 20-500 times larger than the first volume, and the third volume is 10-1,000 times larger than the second volume.

14. The method for preparing high-activity nattokinase powder according to claim 13, wherein in the first-stage fermentation step, the first strain culture solution and the fed-batch feeding culture solution together account for 30%-80% of the second volume; and in the second-stage fermentation step, the second strain culture solution and the fed-batch feeding culture solution together account for 30%-70% of the third volume.

15. The method for preparing high-activity nattokinase powder according to claim 1, wherein the culturing temperatures of the activation step, the first-stage fermentation step and the second-stage fermentation step are 35-40 °C.

16. The method for preparing high-activity nattokinase powder according to claim 1, wherein during the culture process of the first-stage fermentation step and the second-stage fermentation step, the pH value is maintained at 6.5 by using sodium hydroxide and hydrochloric acid, and the dissolved oxygen (DO) is maintained at 5%-50%.

17. The method for preparing high-activity nattokinase powder according to claim 1, wherein in the culture process of the first-stage fermentation step, after 5-hours, the fed-batch feeding culture solution is continuously added into the first strain culture solution using a peristaltic pump for a total fed-batch feeding time of 3-18 hours; and in the culture process of the second-stage fermentation step, after 3-12 hours, the fed-batch feeding culture solution is continuously added into the second strain culture solution using the peristaltic pump for a total fed-batch feeding time of 3-18 hours.

18. The method for preparing high-activity nattokinase powder according to claim 1, wherein in the centrifugation step, the centrifugation conditions are centrifuging at 12,000 rpm and 3-18 °C for 15-60 minutes before taking out.

19. The method for preparing high-activity nattokinase powder according to claim 1, wherein the excipient comprises 8-75 parts of maltodextrin or resistant dextrin mixed with 30-70 parts of trehalose, 15-40 parts of mannitol, 5-15 parts of sorbitol, 5-15 parts of sucrose, 5-15 parts of glycine, 1-10 parts of lactose and 1-parts of dextran. cm 20. The method for preparing high-activity nattokinase powder according to claim 1, wherein the lyophilization step comprises a freezing treatment stage, a primary drying stage and a secondary drying stage sequentially, wherein the freezing treatment stage is freezing at a temperature of −40 °C-0 °C for 2-12 hours, the primary drying stage is slowly raising the temperature from 0 °C to 30 °C for a total time of 10-25 hours after the vacuum pressure is less than 100 mTorr, and the secondary drying stage is drying at a temperature of 30 °C-45 °C for 8-15 hours.
